(19) United States
(12) Patent Application Publication
(10) Pub. No.: US 2006/0287319 A1
(43) Pub. Date: Dec. 21, 2006

(54) Anti-viral compositions comprising heterocyclic substituted phenyl furans and related compounds

(76) Inventors: Shibo Jiang, Fresh Meadows, NY (US); Asim Kumar Debnath, Fort Lee, NJ (US); Hong Lu, Bayside, NY (US)

Correspondence Address: LAW OFFICES OF ALBERT WAI-KIT CHAN, LLC, WORLD PLAZA, SUITE 604, 141-07 20TH AVENUE, WHITESTONE, NY, 11357, US

(21) Appl. No.: 11/448,439

(22) Filed: Jun. 6, 2006

Related U.S. Application Data
(60) Provisional application No. 60/691,120, filed on Jun. 15, 2005.

(51) Int. Cl. A61K 31/53; A61K 31/501; A61K 31/497; A61K 31/496; A61K 31/4439; A61K 31/427

(52) U.S. Cl. 514/241; 514/252.13; 514/255.05; 514/252.02; 514/252.05; 514/369; 514/253.09; 514/341; 514/342

(57) ABSTRACT

A group of compounds that inhibit HIV replication by blocking HIV entry was identified. One representative compound, designated NB-206, and its analogs inhibited HIV replication (p24 production) with IC₅₀ values at nanomolar levels. It was proved that NB-206 and its analogs are HIV entry inhibitors by targeting the HIV gp41 since: 1) they inhibited HIV-mediated cell fusion; 2) they inhibited HIV replication only when they were added to the cells less than one hour after virus addition; 3) they blocked the formation of the gp41 core that is detected by sandwich enzyme linked immunosorbent assay (ELISA) using a conformation-specific MAb NC-1; and 4) they inhibited the formation of the gp41 six-helix bundle revealed by fluorescence native-polyacrylamide gel electrophoresis (FN-PAGE). These results suggested that NB-206 and its analogs may interact with the hydrophobic cavity and block the formation of the fusion-active gp41 coiled coil domain, resulting in inhibition of HIV-1 mediated membrane fusion and virus entry.

This application claims the benefit of U.S. Ser. No. 60/691,120, filed Jun. 15, 2005, the contents of which are incorporated herein in its entirety by reference.

The invention disclosed herein was supported in part by National Institute of Health Grant RO1 AI46221. Accordingly, the United States Government may have certain rights in this invention.

Throughout this application, various publications are referenced and full citations for these publications may be found in the text where they are referenced. Disclosures of these publications in their entireties are hereby incorporated by reference into this application to more fully describe the state of the art to which this invention pertains.

### BACKGROUND OF THE INVENTION

The entry of HIV-1 into host cells is mediated by the binding of the surface subunit gp120 to the host cell receptor CD4. This results in conformational changes and exposure of specific domains on gp120 (1-4). These domains subsequently interact with cellular coreceptors, i.e., CXCR4 or CCR5, leading to the destabilization of the gp120-gp41 complex (5,6). As a result, gp41 undergoes a conformation change exposing the hydrophobic fusion peptide, which inserts into the target cell membrane and initiates the fusion of HIV-1 membranes with the cell membranes (7,8). Therefore, gp41 plays an important role in the early steps of viral entry to the host cells and is considered an important target for developing HIV-1 entry inhibitors. The gp41 molecule consists of three domains, i.e., cytoplasmic domain, transmembrane domain and extracellular domain (ectodomain). The ectodomain contains three major functional regions: the fusion peptide (FP), the N-terminal heptad repeat (NHR or HR1) and the C-terminal heptad repeat (CHR or HR2). The heptad repeat regions generally form typical α-helical structures. Wild et al (9,10) and Jiang et al (11), about a decade ago, showed that peptides from the HR1 and HR2 regions inhibit HIV-1 infection at low nanomolar concentrations. This resulted in the discovery of the entry inhibitor, T-20 (Fuzeon, Enfuvirtide), which was approved by the US FDA in 2003 as an anti-HIV-1 drug (12,13). This also provided a direct proof of the concept that disrupting six-helix bundle formation is a valid strategy for developing antiviral agents. Discovery of this drug is a great breakthrough in the development of anti-HIV drugs since it can be used for treatment of HIV-infected individuals who fail to respond to the currently available anti-retroviral drugs, such as HIV reverse transcriptase and protease inhibitors (14,15). However, the future application of T-20 may be constrained due to its lack of oral availability and high cost of production. Therefore, it is essential to develop small molecule anti-HIV-1 compounds with a mechanism of action similar to that of C-peptides but without the disadvantages of the peptidic drugs.

Research on the mechanism by which C-peptides inhibit HIV-1 fusion has demonstrated that gp41 N— and C-peptides mixed at equimolar concentrations form a stable α-helical trimer of antiparallel heterodimers, representing the fusion-active gp41 core (16,17). The crystallographic data of the core revealed that NHR peptides form an inner trimeric coiled-coil consisting of hydrophobic grooves and that the CHR peptides fold back in an anti-parallel fashion to form a stable hairpin-like structure called a six-helix bundle (7,18). This stable six-helix bundle formation is thought to bring the viral membranes and host cell membranes together, a prerequisite for membrane fusion. The six-helix bundle formation has been recently reported to be a necessary step to form fusion pores (19). Therefore, disruption of the six-helix bundle formation by targeting the hydrophobic grooves has been recognized as a strategy to develop antiviral agents (7,20). Several small molecule compounds were identified using gp41 pocket as the target structure, e.g., ADS-J1 (21,22), XTT formazan (23), NB-2 and NB-64 (24). A combination of techniques were used in those studies, e.g., a cell-based HIV fusion assay (25,26), a sandwich enzyme linked immunosorbent assay (ELISA) (27) and a fluorescence enzyme linked immunosorbent assay (FLISA) (28) using a monoclonal antibody (mAb), NC-1, which specifically recognizes the fusion-active gp41 core structure (29) and computer-aided molecular docking technique (21). These compounds inhibit HIV-1 fusion possibly by docking into the gp41 pocket and interfering with the formation of the gp41 six-helix bundle formation. However, they may not be good lead compounds for development of anti-HIV-1 drugs since their anti-HIV-1 activity is not very potent (IC₅₀ values are in micromolar level) Nevertheless, the identification of these compounds is useful as a proof of concept that a small molecule organic compound might block the fusion-active gp41 six-helix bundle formation and inhibit HIV-1 entry or the entry of other viruses. Here we report the identification of a series of derivatives of 3-[5-(2,4-dioxo-thiazolidin-ylidenemethyl)-furan-2-yl]-benzoic acid and 3-[5-(4-oxo-2-thioxo-thiazolidin-ylidenemethyl)-furan-2-yl]-benzoic acid, represented by NB-206 and its analogs, as anti-viral compositions, e.g., novel HIV-1 fusion inhibitors. These compounds may interact with gp41 at the fusion-intermediate conformation, possibly by binding to the gp41 hydrophobic pocket and surrounding area and blocking the gp41 six-helix bundle formation, thereby inhibiting the fusion between the viral and target cell membranes. NB-206 and its analogs are “drug-like” compounds and may be used as leads for designing more potent anti-virus compositions, e.g. HIV-1 entry inhibitors, which are expected to be developed as a new class of anti-viral, e.g., anti-HIV-1, drugs.

### SUMMARY OF THE INVENTION

It is an object of the present invention to provide compounds and compositions, which are effective against HIV infection.

It is also an object of the present invention to provide compounds and compositions for design and development of a new class of anti-HIV drugs by blocking HIV entry.

It is a further object of the present invention to provide methods for inhibiting HIV replication or infectivity or treating HIV infection in a subject without inducing undesirable adverse effects.

The present invention comprises compounds of the formula I, or pharmaceutically acceptable salts thereof,

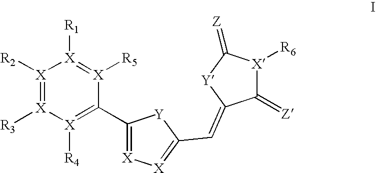

Wherein at least one of R₁, R₂ R₃, R₄, R₅, or R₆ contains COOH or other acidic groups.

X and X′,Y and Y′ can be either C, N, O or S and Z and Z′ can be O or S. When X and X′,Y and Y′ are either O or S, the bond with the next atom such as C, will be a single bond and O or S will be unsubstituted and when X and X′,Y and Y′ are N, it is either unsubstituted or substituted with H, alkyl, cycloalkyl, alkenyl, alkynyl, aryl, arylalkyl or heterocyclyl groups. In an embodiment, R₁—R₆ are independently selected from the groups consisting of, but not limited to, H, alkyl, cycloalkyl, alkenyl, alkynyl, aryl, arylalkyl, alkylaryl, heterocyclyl, tetrazolyl, adamantyl, halogen, trifluoromethyl, OH, CN, NO₂ and OR₇, where R₇ is alkyl, aryl, or arylalkyl, COOR₈, where R₈ is H and alkyl, SO₃R₉, where R₉ is H and alkyl, SO₂NHR₁₀, where R₁₀ is H and alkyl, and CONHR₁₁ where R₁₁ is H or alkyl.

The group alkyl is represented by optionally substituted straight or branched alkyl chains carrying 1 to 6 carbon atoms and accordingly preferably stands for methyl, ethyl, n-propyl, i-propyl, n-butyl, i-butyl, tert-butyl, pentyl or hexyl.

The group alkenyl is represented by optionally substituted straight or branched alkenyl chains carrying 2 to 6 carbon atoms and accordingly preferably stands for vinyl, 1-propenyl, 2-propenyl, i-propenyl, butenyl and its isomers, pentenyl or hexenyl.

The group alkynyl is represented by optionally substituted straight or branched alkynyl chains carrying 2 to 6 carbon atoms and accordingly preferably stands for ethynyl, propynyl and its isomers, butynyl and its isomers, pentynyl or hexynyl.

Suitable substituents of alkyl, alkenyl and alkynyl can be selected from one or more of amino, cyano, halogen, hydroxy, alkoxy, aryloxy, aryl, heterocyclyl, carboxy, nitro, alkyl sulfonyl, aryl sulfonyl, thio, alkyl thio, aryl thio.

The group cycloalkyl is represented by optionally substituted cycloalkyl groups containing 3 to 6 carbon atoms and can be selected, e.g., cyclopropyl, cyclobutyl, cyclopentyl, cyclohexyl, or adamantyl. All these groups can also be benz-fused to an aromatic cyclic group, e.g., phenyl.

The group aryl is represented by optionally substituted phenyl or napthyl. In an embodiment, both phenyl or napthyl are optionally substituted with amino, cyano, halogen, hydroxyl, alkoxy, carboxy, nitro, thio, alkyl, or trifluoromethyl.

The group heterocyclic stands for optionally substituted saturated, partially saturated, aromatic cyclics, which contain one or more heteroatoms selected from nitrogen, oxygen and sulfur and can also be benz-fused to an optionally substituted aromatic cyclic or heterocyles.

Heterocyclic groups can be selected, but not limited to, from quinolinyl, pyridyl, indolyl, furyl, oxazolyl, thienyl, triazolyl, pyrazolyl, imidazolyl, benzothiazolyl, benzimidazolyl, piperzinyl, benzothiazolyl.

Substituents for aryl and heterocyclyl can be selected from those mentioned for alkyl.

The group halogen stands for chloro, bromo, fluoro and iodo.

Compounds of formula I, which have acid groups can form pharmaceutically acceptable salts with inorganic and organic bases, e.g., sodium hydroxide, potassium hydroxide, calcium hydroxide, barium hydroxide, magnesium hydroxide, N-ethyl piperidine, and similar other bases. When formula I is basic in nature it can form pharmaceutically acceptable salts with inorganic and organic acids, e.g., hydrochloric acid, sulfuric acid, nitric acid, phosphoric acid, acetic acid, tartaric acid, succinic acid, fumaric acid, maleic acid, malic acid, citric acid, methane sulfonic acid and similar others acids.

Any compounds, compositions, or embodiments comprising formula I may exist as stereoisomers, e.g., E- or Z-isomers.

TABLE 1
Structures and molecular weights of NB-206 and its analogs

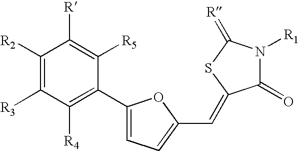

| No. | Code | R′ | R″ | R₁ | R₂ | R₃ | R₄ | R₅ | MW |
|---|---|---|---|---|---|---|---|---|---|
| 1 | NB-139 | 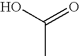 | O | 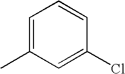 | Cl | H | H | H | 460.30 |
| 2 | NB-140 | 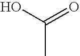 | O | 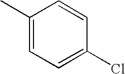 | Cl | H | H | H | 460.30 |
| 3 | NB-145 | 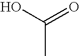 | S | 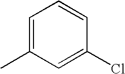 | H | H | H | H | 441.92 |
| 4 | NB-146 | 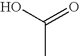 | S | 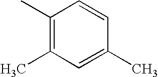 | H | H | H | H | 435.52 |
| 5 | NB-147 | 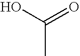 | S | 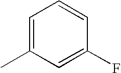 | H | H | H | H | 425.46 |
| 6 | NB-148 | 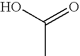 | S | 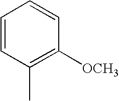 | H | H | H | H | 437.50 |
| 7 | NB-150 | 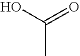 | S | 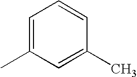 | H | H | H | H | 421.50 |
| 8 | NB-151 | 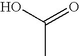 | S | CH₃ | H | H | H | H | 345.40 |
| 9 | NB-154 | 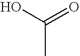 | S | 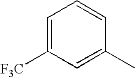 | H | H | H | H | 475.47 |
| 10 | NB-156 | 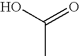 | S | 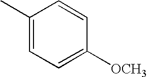 | H | H | H | H | 437.50 |
| 11 | NB-158 | 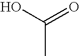 | O | 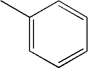 | H | H | H | H | 391.41 |
| 12 | NB-160 | 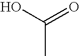 | O | 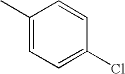 | H | H | H | H | 425.85 |
| 13 | NB-179 | 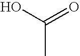 | S | CH₂CH₃ | H | H | H | H | 359.43 |
| 14 | NB-180 | 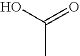 | S | CH₂COOH | H | H | H | H | 389.41 |
| 15 | NB-181 | 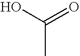 | S | 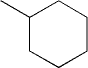 | H | H | H | H | 413.52 |
| 16 | NB-182 | 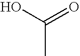 | S | 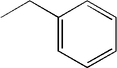 | H | H | H | H | 421.50 |
| 17 | NB-183 | 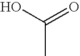 | S | CH₂CH═CH₂ | H | H | H | H | 371.44 |
| 18 | NB-184 | 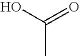 | S | H | H | H | H | H | 331.37 |
| 19 | NB-185 | 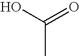 | S | H | Cl | H | H | H | 365.82 |
| 20 | NB-186 | 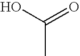 | O | CH₃ | H | H | Cl | H | 363.78 |
| 21 | NB-187 | 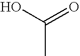 | O | 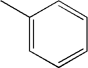 | H | H | CH₃ | H | 405.43 |
| 22 | NB-188 | 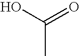 | S | H | H | H | CH₃ | H | 345.40 |
| 23 | NB-189 | 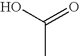 | S | CH₂CH═CH₂ | H | H | H | CH₃ | 385.46 |
| 24 | NB-190 | 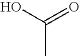 | S | H | H | H | Cl | H | 365.82 |
| 25 | NB-191 | 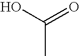 | O | 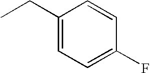 | Cl | H | H | H | 457.87 |
| 26 | NB-192 | 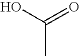 | O | 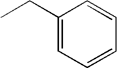 | Cl | H | H | H | 439.88 |
| 27 | NB-193 | 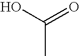 | O | CH₂CH(CH₃)₂ | Cl | H | H | H | 405.86 |
| 28 | NB-194 | 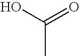 | O | 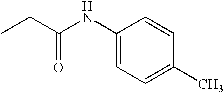 | H | H | H | H | 462.48 |
| 29 | NB-195 | 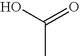 | O | 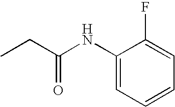 | H | H | H | CH₃ | 480.48 |
| 30 | NB-196 | 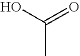 | O | 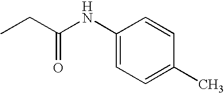 | H | H | H | CH₃ | 476.51 |
| 31 | NB-197 | 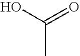 | O | 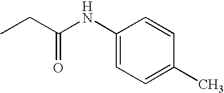 | H | H | Cl | H | 496.93 |
| 32 | NB-198 | 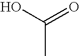 | O | 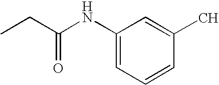 | H | H | Cl | H | 496.93 |
| 33 | NB-199 | 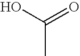 | O | 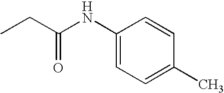 | Cl | H | H | H | 496.93 |
| 34 | NB-200 | 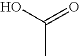 | O | CH₂CH≡CH | Cl | H | H | H | 387.80 |
| 35 | NB-201 | 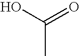 | S | CH₂CH₂OCH₃ | Cl | H | H | H | 423.90 |
| 36 | NB-202 | 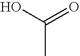 | O | 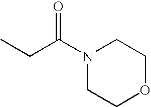 | Cl | H | H | H | 476.90 |
| 37 | NB-203 | 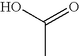 | O | CH₂CH≡CH | H | H | Cl | H | 387.80 |
| 38 | NB-204 | 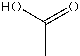 | O | 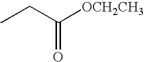 | Cl | H | H | H | 435.84 |
| 39 | NB-205 | 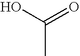 | O | CH₂CH≡CH | H | H | CH₃ | H | 367.38 |
| 40 | NB-206 | 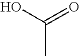 | S | 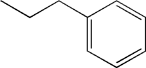 | Cl | H | H | H | 469.97 |
| 41 | NB-207 | 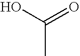 | O | 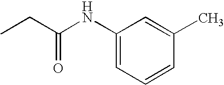 | Cl | H | H | H | 496.93 |
| 42 | NB-208 | 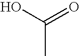 | S | 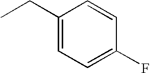 | Cl | H | H | H | 473.93 |
| 43 | NB-209 | 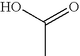 | O | 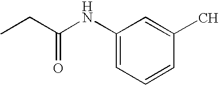 | H | H | H | CH₃ | 476.51 |
| 44 | NB-210 | 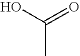 | S | 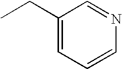 | Cl | H | H | H | 456.93 |
| 45 | NB-211 | 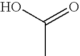 | S | 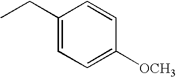 | Cl | H | H | H | 485.97 |
| 46 | NB-212 | 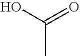 | O | 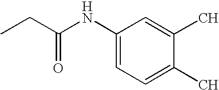 | H | H | CH₃ | H | 490.54 |
| 47 | NB-213 | 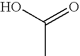 | S | 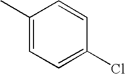 | Cl | H | H | H | 476.36 |
| 48 | NB-214 | 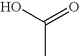 | S | CH₂CH₃ | Cl | H | H | H | 393.87 |
| 49 | NB-215 | 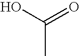 | S | 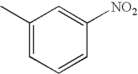 | Cl | H | H | H | 486.91 |
| 50 | NB-216 | 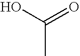 | O | CH₂CH≡CH | H | H | H | H | 353.36 |
| 51 | NB-217 | 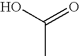 | S | 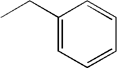 | Cl | H | H | H | 455.94 |
| 52 | NB-218 | 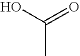 | S | 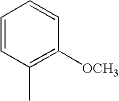 | H | H | H | H | 421.50 |
| 53 | NB-219 | 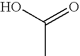 | S | 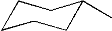 | Cl | H | H | H | 447.96 |
| 54 | NB-220 | 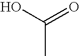 | S | 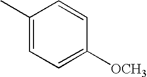 | Cl | H | H | H | 471.94 |
| 55 | NB-221 | 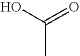 | S | 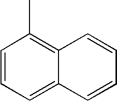 | H | H | H | H | 457.53 |
| 56 | NB-222 | 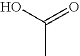 | S | 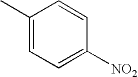 | Cl | H | H | H | 486.91 |
| 57 | NB-223 | 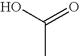 | S | 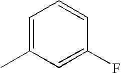 | Cl | H | H | H | 459.91 |
| 58 | NB-224 | 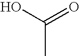 | S | 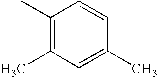 | Cl | H | H | H | 469.97 |
| 59 | NB-225 | 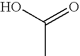 | S | CH₂CH═CH₂ | H | COOH | H | H | 415.45 |
| 60 | NB-226 | 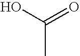 | S | 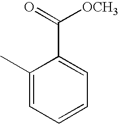 | Cl | H | H | H | 499.95 |
| 61 | NB-227 | 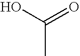 | S | 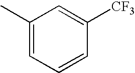 | H | H | H | CH₃ | 489.50 |
| 62 | NB-228 | 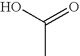 | S | 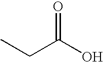 | H | H | H | CH₃ | 403.44 |
| 63 | NB-229 | 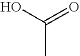 | S | 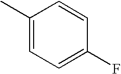 | H | H | H | H | 425.46 |
| 64 | NB-230 | 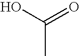 | S | 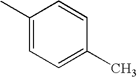 | H | H | H | H | 421.50 |
| 65 | NB-231 | 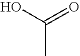 | S | 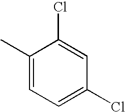 | H | H | H | H | 476.36 |
| 66 | NB-232 | 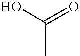 | O | CH(CH₃)CH₂CH₃ | H | H | Cl | H | 405.86 |
| 67 | NB-233 | 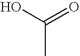 | O | 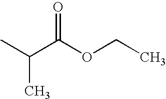 | H | H | CH₃ | H | 429.45 |
| 68 | NB-234 | 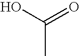 | O | 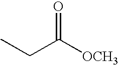 | H | H | Cl | H | 421.82 |
| 69 | NB-235 | 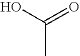 | S | 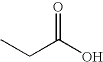 | H | H | Cl | H | 423.85 |
| 70 | NB-236 | 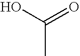 | O | CH₂CH₃ | H | H | Cl | H | 377.81 |
| 71 | NB-237 | 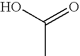 | O | 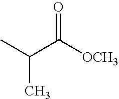 | H | H | CH₃ | H | 415.42 |
| 72 | NB-238 | 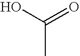 | S | 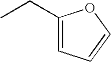 | H | H | H | H | 411.46 |
| 73 | NB-239 | Cl | S | 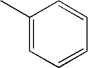 | H | H | H | H | 397.90 |

A synthetic peptide drug, T-20, has shown potent anti-HIV activity by blocking HIV entry in clinical trial. However, its future clinical application will be limited due to lack of oral availability. A group of organic compounds with low molecular weight having potent anti-HIV activity were identified by blocking HIV entry with a mechanism of action similar to that of T-20. We found that NB-206 and its analogs, inhibited HIV replication (p24 production), HIV-mediated cytopathic effect (CPE) and cell fusion with low IC₅₀ values (Table 2). It was proved that NB-206 and its analogs are HIV entry inhibitors by targeting the HIV gp41 since: 1) they inhibited HIV-mediated cell fusion; 2) they inhibited HIV replication only when they were added to the cells less than two hours after Virus addition; 3) they blocked the formation of the gp41 core detected by sandwich enzyme linked immunosorbent assay (ELISA) using a conformation-specific MAb NC-1; and 4) they inhibited the formation of the gp41 six-helix bundle revealed by fluorescence native-polyacrylamide gel electrophoresis (FN-PAGE). These results suggested that NB-206 and its analogs may interact with the hydrophobic cavity and block the formation of the fusion-active gp41 coiled coil domain, resulting in inhibition of HIV-1 mediated membrane fusion and virus entry.

### DETAILED DESCRIPTION OF THE FIGURES

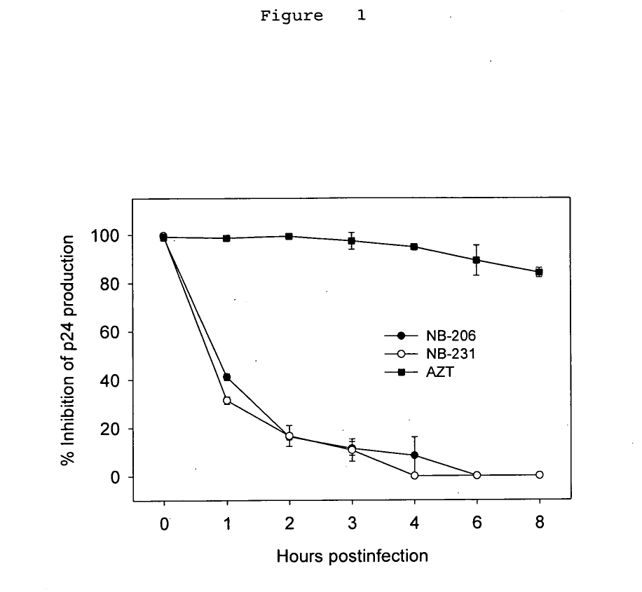

FIG. 1 NB-206 and its analogs inhibited HIV-1 entry. Inhibition of HIV-1 entry was determined by a time-of-addition assay. NB-206 (2.5 μM) and its analog NB-231 (2.5 μM) were added to MT-2 cells at different intervals post-infection by HIV-1_{IIIB}. AZT (0.1 μM), a reverse transcriptase inhibitor, was included as a control. Each sample was tested in triplicate.

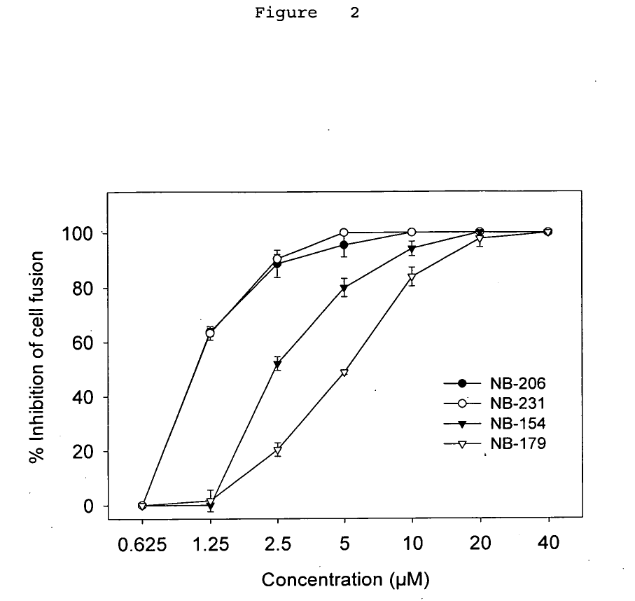

FIG. 2 NB-206 and its analogs inhibited HIV-1 mediated cell-cell fusion. Inhibition of fusion between HIV-1_{IIIB} infected H9 cells (H9/HIV-1_{IIIB}) labeled with Calcein and MT-2 cells were assessed by a dye transfer assay as described in the Materials and Methods. Each sample was tested in quadruplicate.

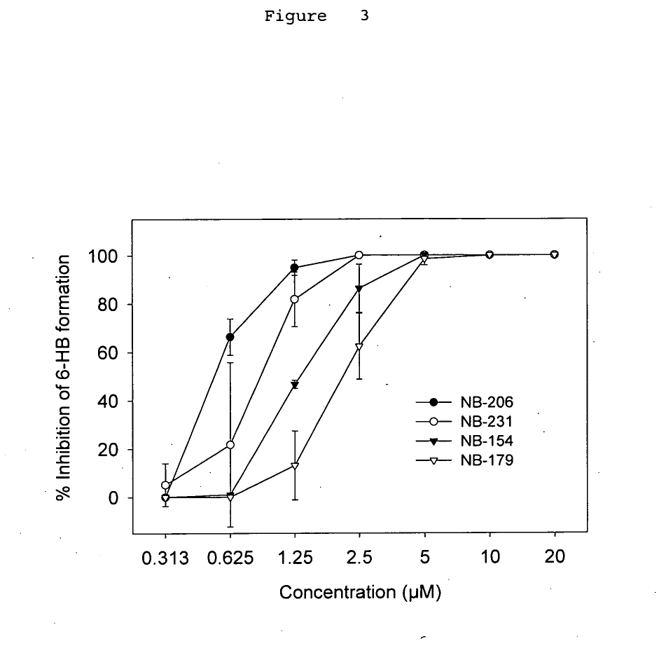

FIG. 3 NB-206 and its analogs inhibited the gp41 six-helix bundle formation as measured by a sandwich ELISA (A) and FN-PAGE. The compounds NB-206 and its analogs were incubated with N36 for 30 min at 37° C. before addition of C34. Samples were tested in triplicate in ELISA.

### DETAILED DESCRIPTION OF THE INVENTION

Screening methods of antiviral compounds targeted to the HIV-1 gp41 core structure were described in Patent Cooperation Treaty (PCT) application, PCT/US00/06771, publication no. WO 00/55377, U.S. Pat. No. 6,596,497. PCT application, PCT/US2003/036359, publication W02004/047730, further describes that antiviral compounds may be screened by the following method:
a) capturing polyclonal antibodies from an animal other than a mouse, directed against the HIV-1 gp41 trimeric structure containing three N-peptides of HIV-1 gp41 and three C-peptides of HIV-1 gp41, onto a solid-phase to form a polyclonal antibody coated solid-phase;
b) forming a mixture of a compound to be tested with N-peptides of HIV-1 gp41, and then-adding C-peptides of HIV-1 gp41;
c) adding the mixture from step (b) to the polyclonal antibody coated solid-phase from step (a), then removing unbound peptides and unbound compound, and then adding a monoclonal antibody which specifically reacts with the HIV-1 gp41 and three C-peptides of HIV-1 gp41, but does not react with individual N-peptides of HIV-1 gp41 and does not react with individual C-peptides of HIV-1 gp41; and
d) measuring the binding of said monoclonal antibody.

The monoclonal antibody used in screenings is designated NC-1. A biological assay may be used with the above immunoscreening assay. Said biological assay includes but is not limited to HIV-mediated cell fusion assay, as described infra. The assay may also be fluorescence native polyacrylamide gel electrophoresis (FN-PAGE).

As a result of the screening, some lead components were identified.

This invention comprises an effective amount of a compound comprising formula (I) or a pharmaceutically acceptable salt thereof:

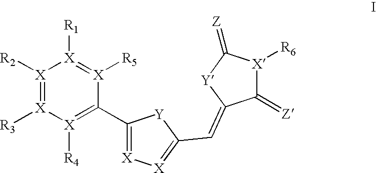

Wherein at least one of R₁, R₂ R₃, R₄, R₅, or R₆ contains COOH or other acidic groups.

X and X′,Y and Y′ can be either C, N, O or S and Z and Z′ can be O or S. When X and X′,Y and Y′ are either O or S, the bond with the next atom such as C, will be a single bond and O or S will be unsubstituted and when X and X′,Y and Y′ are N, it is either unsubstituted or substituted with H, alkyl, cycloalkyl, alkenyl, alkynyl, aryl, arylalkyl, or heterocyclyl groups. In an embodiment, R₁-R₆ are independently selected from the groups consisting of, but not limited to, H, alkyl, cycloalkyl, alkenyl, alkynyl, aryl, arylalkyl, heterocyclyl, tetrazolyl, adamantyl, halogen, trifluoromethyl, OH, CN, NO₂ and OR₇, where R₇ is alkyl, aryl, or arylalkyl, COOR₈, where R₈ is H and alkyl, SO₃R₉, where R₉ is H and alkyl, SO₂NHR₁₀, where R₁₀ is H and alkyl, and CONHR₁₁ where R₁₁ is H or alkyl.

This invention provides a compound having formula I, wherein X is a carbon, X′ is nitrogen, Y and Z′ are oxygen, Y′ and Z are sulfur, or its pharmaceutically acceptable salts,

Wherein:

R₁—R₆ is independently selected from the group consisting of, but not limited to, H, alkyl, cycloalkyl, alkenyl, alkynyl, aryl, arylalkyl, alkylaryl, heterocyclyl, tetrazolyl, adamantyl, halogen, trifluoromethyl, OH, CN, NO₂ and OR₇, where R₇ is alkyl, aryl, or arylalkyl, COOR₈, where R₈ is H and alkyl, SO₃R₉, where R₉ is H and alkyl, SO₂NHR₁₀, where R₁₀ is H and alkyl, and CONHR₁₁ where R₁₁ is H or alkyl.

In an embodiment, the group alkyl is substituted with straight or branched alkyl chains carrying 1 to 6 carbon atoms.

In another embodiment, alkyl is methyl, ethyl, n-propyl, i-propyl, n-butyl, i-butyl, tert-butyl, pentyl or hexyl.

In a separate embodiment, alkenyl is substituted with straight or branched alkenyl chains carrying 2 to 6 carbon atoms. The alkenyl includes but is not limited to vinyl, 1-propenyl, 2-propenyl, i-propenyl, butenyl, or its isomers, pentenyl or hexenyl.

In an embodiment, alkynyl is substituted with straight or branched alkynyl chains carrying 2 to 6 carbon atoms. The alkynyl group includes but is not limited to ethynyl, propynyl or its isomers, or butynyl or its isomers, pentynyl or hexynyl.

In accordance with this invention, suitable substituents of alkyl, alkenyl and alkynyl can be selected from one or more of the following: amino, cyano, halogen, hydroxy, alkoxy, aryloxy, aryl, heterocyclyl, carboxy, nitro, alkyl sulfonyl, aryl sulfonyl, thio, alkyl thio, or aryl thio.

In an embodiment, this invention provides the above compound, wherein cycloalkyl is substituted with cycloalkyl groups containing 3 to 6 carbon atoms. The cycloalkyl includes but not limited to cyclopropyl, cyclobutyl, cyclopentyl, cyclohexyl, and adamantyl. In a further embodiment, the cycloalkyl is benz-fused to an aromatic cyclic group.

In a separate embodiment, the aryl is substituted with phenyl or napthyl (both optionally substituted with amino, cyano, halogen, hydroxyl, alkoxy, carboxy, nitro, thio, alkyl, or trifluoromethyl).

This invention provides the above compound, wherein the group heterocyclic is optionally substituted with saturated, partially saturated, or aromatic cyclics, which contain one or more heteroatoms selected from nitrogen, oxygen or sulfur. In an embodiment, the compound is benz-fused to a substituted aromatic cyclic or heterocyles. In a further embodiment, the heterocyclic group includes but is not limited to quinolinyl, pyridyl, indolyl, furyl, oxazolyl, thienyl, triazolyl, pyrazolyl, imidazolyl, benzothiazolyl, benzimidazolyl, piperzinyl, and benzothiazolyl.

This invention provides the above compound, wherein the halogen group is chloro, bromo, fluoro, or iodo.

This invention provides a compound having formula I, which has acid group(s) and capable of forming pharmaceutically acceptable salts with inorganic and organic bases. The base includes but is not limited to sodium hydroxide, potassium hydroxide, calcium hydroxide, barium hydroxide, magnesium hydroxide, and N-ethyl piperidine.

This invention provides a compound having formula I, wherein X is a carbon, XI is nitrogen, Y and Z′ are oxygen, Y′ and Z are sulfur, and R₁ is COOH, R₂ is chloro, R₃—R₅ are hydrogen, R₆ is propylbenzene.

Any compounds, compositions, or embodiments comprising formula I may exist as stereoisomers, e.g., E- or Z-isomers.

This invention provides an antiviral pharmaceutical composition comprising an effective amount of a compound with formula I, or a pharmaceutically acceptable salt, and a pharmaceutically acceptable carrier.

A “pharmaceutically acceptable carrier” means any of the standard pharmaceutical carriers. Examples of suitable carriers are well known in the art and may include but are not limited to any of the standard pharmaceutical carriers like phosphate buffered saline solutions, phosphate buffered saline containing Polysorb 80, water, emulsions such as oil/water emulsion, and various types of wetting agents. Other carriers may also include sterile solutions, tablets, coated tablets, and capsules.

Typically such carriers contain excipients like starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

This invention provides the above pharmaceutical composition for treating human immunodeficiency virus (HIV) infection, further comprising an effective amount of an Acquired Immunodeficiency Syndrome (AIDS) treatment agent selected from the group consisting of anti-HIV agents, anti-infective agents, and immunomodulators.

This invention provides a method for inhibiting replication of human immunodeficiency virus in cells comprising of contacting cells with an effective amount of a compound with formula I to inhibit the replication of the human immunodeficiency virus.

This invention provides a method for treating a subject infected with the human immunodeficiency virus, comprising administering to said subject an effective amount of a compound with formula I, or its pharmaceutically acceptable salts thereof.

This invention provides a method for preventing manifestation of Acquired Immunodeficiency Syndrome (AIDS) in a subject comprising administering to the subject an amount of a compound with formula I effective to prevent said syndrome in the subject.

In an embodiment of the above method, the subject is a human.

The invention will be better understood by reference to the Examples which follow, but those skilled in the art will readily appreciate that the specific examples are only illustrative and are not meant to limit the invention as described herein, which is defined by the claims which follow thereafter.

Experimental Details

Materials and Methods

Reagents. MT-2 cells, HIV-1_{IIIB}-infected H9 cells (H9/HIV-1_{IIIB}), U87-T4-CXCR4 and U87-T4-CCR5 cells, laboratory adapted and primary HIV-1 strains, and anti-p24 mAb (183-12H-5C) were obtained from the NIH AIDS Research and Reference Reagent Program. Lymphoid cell line CEMx174 5.25M7, kindly provided by C. Cheng-Mayer, is stably transduced with an HIV-1 long terminal repeat (LTR)-green fluorescent protein (GFP) reporter and luciferase reporter construct. The cells express CD4 and both coreceptors, CXCR4 and CCR5 (30). These cells were maintained in RPMI-1640 medium supplemented with 10% FBS, 1 μg/ml puromycin, 200 μg/ml G418. Recombinant soluble CD4 (sCD4) was obtained from Genentech Inc. (South San Francisco, Calif.). Peptides N36, C34 (7,17), IQN17 (31), and T22 (32,33) were synthesized by a standard solid-phase FMOC method in the MicroChemistry Laboratory of the New York Blood Center. A biotinylated D-peptide, D10-p5-2K (31), was also synthesized in-house with D-amino acids and was oxidized as previously described (31). The peptides were purified to homogeneity by high-performance liquid chromatography (HPLC). The identity of the purified peptides was confirmed by laser desorption mass spectrometry (PerSeptive Biosystems). Rabbit antisera directed against the mixture of N36/C34 and against IQN17 were prepared as previously described (29). Mouse mAb NC-1 specific for the gp41 six-helix bundle was prepared and characterized as previously described (29). Rabbit and mouse IgG were purified using Protein A/G beads (Pierce, Rockford, Ill.). Mouse mAb 12G5 specific for CXCR4 was purchased from R&D Systems (Minneapolis, Minn.). The chemical library used for screening was purchased from Nanosyn (Menlo Park, Calif.). NB-206 and its analogs were purchased from ChemBridge Corporation (San Diego, Calif.). Chloropeptin was a generous gift from Satoshi Omura and Haruo Tanaka of The Kitasato Institute, Tokyo, Japan.

Syncytium-formation assay for screening HIV-1 fusion inhibitors. HIV-1_{IIIB}-infected H9 cells (H9/HIV-1_{IIIB}) at 2×10⁵/ml were cocultured with MT-2 cells (2×10⁶/ml) in the presence of compounds to be screened (final concentration of compound: 25 μg/ml) in a 96-well plate at 37° C. for 2 days. HIV-1 induced syncytium formation was observed under an inverted microscope and scored as “−” (no syncytium was observed), “±” (about 50% syncytia were inhibited), and “+” (no syncytium formation was inhibited). The compounds scored with “−” and “±” were selected for further screening by ELISA for inhibitors against the gp41 six-helix bundle formation.

ELISA for screening for compounds that inhibit the gp41 six-helix bundle formation. A sandwich ELISA as previously described (27) was used to screen for compounds that inhibit the gp41 six-helix bundle formation. Briefly, peptide N36 (2 μM) was pre-incubated with a test compound at the indicated concentrations at 37° C. for 30 min, followed by addition of C34 (2 μM). In the control experiments, N36 was pre-incubated with C34 at 37° C. for 30 min, followed by addition of the test compound. After incubation at 37° C. for 30 min, the mixture was added to wells of a 96-well polystyrene plate (Costar, Corning Inc., Corning, N.Y.) which were precoated with IgG (2 μg/ml) purified from rabbit antisera directed against the N36/C34 mixture. Then, the mAb NC-1, biotin-labeled goat-anti-mouse IgG (Sigma Chemical Co., St. Louis, Mo.), streptavidin-labeled horseradish peroxidase (SA-HRP) (Zymed, S. San Francisco, Calif.), and the substrate 3,3′,5,5′-tetramethylbenzidine (TMB) (Sigma) were added sequentially. Absorbance at 450 nm was measured using an ELISA reader (Ultra 384, Tecan, Research Triangle Park, N.C.). The percent inhibition by the compounds was calculated as previously described (34) and the concentration for 50% inhibition (IC₅₀) was calculated using the software designated Calcusyn (35), kindly provided by Dr. T. C. Chou (Sloan-Kettering Cancer Center, New York, N.Y.).

Assessment of anti-HIV-1 infectivity. The inhibitory activity of compounds on infection by laboratory-adapted HIV-1 strains was determined as previously described (34). In brief, 1×10⁴ MT-2 cells were infected with HIV-1 at 100 TCID₅₀ (50% tissue culture infective dose) in 200 μl of RPMI 1640 medium containing 10% FBS in the presence or absence of compounds at graded concentrations overnight. For the time-of-addition assay, compounds were added at various time post-infection. Then the culture supernatants were removed and fresh media were added. On the fourth day post-infection, 100 μl of culture supernatants were collected from each well, mixed with equal volumes of 5% Triton X-100 and assayed for p24 antigen, which was quantitated by ELISA (23). Briefly, the wells of polystyrene plates (Immulon 1B, Dynex Technology, Chantilly, Va.) were coated with HIV immunoglobulin (HIVIG), which was prepared from plasma of HIV-seropositive donors with high neutralizing titers against HIV-1_{IIIB} as previously described (36) in 0.085 M carbonate-bicarbonate buffer (pH 9.6) at 4° C. overnight, followed by washes with PBS-T buffer (0.01M PBS containing 0.05% Tween-20) and blocking with PBS containing 1% dry fat-free milk (Bio-Rad Inc., Hercules, Calif.). Virus lysates were added to the wells and incubated at 37° C. for 1 h. After extensive washes, anti-p24 mAb (183-12H-5C), biotin labeled anti-mouse IgG1 (Santa Cruz Biotech., Santa Cruz, Calif.), SA-HRP and TMB were added sequentially. Reactions were terminated by addition of 1N H₂SO₄. Absorbance at 450 nm was recorded in an ELISA reader (Ultra 384, Tecan). Recombinant protein p24 (US Biological, Swampscott, Mass.) was included for establishing standard dose response curve.

Inhibitory activity of compounds on infection by primary HIV-1 isolates was determined as previously described (37). PBMCs were isolated from the blood of healthy donors at the New York Blood Center by standard density gradient centrifugation using Histopaque-1077 (Sigma). The cells were plated in 75 cm² plastic flasks and incubated at 37° C. for 2 hrs. The nonadherent cells were collected and resuspended at 5×10⁶ in 10 ml RPMI-1640 medium containing 10% FBS, 5 μg/ml PHA and 100 U/ml IL-2 (Sigma), followed by incubation at 37° C. for 3 days. The PHA-stimulated cells were infected with corresponding primary HIV-1 isolates at 0.01 multiplicity of infection (MOI) in the absence or presence of a compound at graded concentrations. Culture media were changed every 3 days. The supernatants were collected 7 days post-infection and tested for p24 antigen by ELISA as described above. The percent inhibition of p24 production and IC₅₀ values were calculated as described above.

Inhibition of cell-cell fusion. A dye transfer assay was used for detection of HIV-1 mediated cell fusion as previously described (11,25,26). H9/HIV-1_{IIIB} cells were labeled with a fluorescent reagent, Calcein-AM (Molecular Probes, Inc., Eugene, Oreg.) and then incubated with MT-2 cells (ratio=1:5) in 96-well plates at 37° C. for 2 hrs in the presence or absence of compounds tested. The fused and unfused Cacein-labeled HIV-1-infected cells were counted under an inverted fluorescence microscope (Zeiss, Germany) with an eyepiece micrometer disc. The percentage of inhibition of cell fusion and the IC₅₀ values were calculated as previously described (11).

Inhibition of fusion between PBMCs infected by primary HIV-1 strains (X4 and R5 viruses) with CEMx174 5.25 M7 cells, which express CD4 and both coreceptors, CXCR4 and CCR5, was determined by a luciferase assay. Briefly, 50 μl of compound at graded concentration in triplicate was incubated with equal volume of PHA-stimulated PBMCs (1×10⁵/ml) infected by corresponding primary HIV-1 strains, respectively, for 7 days as described above. After incubation at 37° C. for 30 min, 100 μl of CEMx174 5.25 M7 cells (2×10⁵) were added and incubated at 37° C. for three days. The cells were collected, washed, and lysed with the lysing reagent included in the luciferase kit (Promega, Corp., Madison, Wis.). Aliquots of cell lysates were transferred to 96-well flat-bottom luminometer plates (Costar, Corning Inc., Corning, N.Y.), followed by addition of luciferase substrate (Promega). The luciferase activity was measured in the Ultra 384 luminometer (Tecan).

Detection of in vitro cytotoxicity. The in vitro cytotoxicity of compounds for MT-2 cells was measured by a colorimetric method using XTT tetrazolium dye as previously described (21). Briefly, 100 μl of a compound at a graded concentration was added to equal volume of cells (5×10⁵/ml) in a well of 96-well plates. After incubation at 37° C. for 4 days, XTT (1 mg/ml; 50 ml/well; PolySciences, Inc., Warrington, Pa.) was added. Four hours later, the soluble intracellular formazan was quantitated colorimetrically at 450 nm with a reference at 570 nm. The percent of cytotoxicity (37) and the CC₅₀ (the concentration for 50% cytotoxicity) values were calculated using the software Calcusyn (35).

Inhibition of gp120 binding to CD4. Wells of polystyrene plates was coated with 100 μl of sheep anti-gp120 antibody D7324 (Cliniqa, Fallbrook, Calif.) at 2 μg/ml in carbonate buffer (pH 9.6) at 4° C. overnight and blocked with 1% dry fat-free milk in PBS at 37° C. for 1 h. One hundred microliters of recombinant gp120 molecule (Immunodiagnostics, Woburn, Mass.) at 0.5 μg/ml in PBS was added and incubated at 37° C. for 1 h, followed by three washes with PBS-T. Soluble CD4 (sCD4) at 0.25 μg/ml was added in the presence of a compound (25 μM) and incubated at 37° C. for 1 h. After three washes, rabbit anti-sCD4 IgG (0.25 μg/ml in PBS, 100 μl/well) was added and incubated at 37° C. for 1 h. Binding of rabbit anti-sCD4 IgG was determined by sequential addition of biotinylated goat-anti-rabbit IgG, SA-HRP, and TMB. After the reactions were terminated, absorbance at 450 nm was recorded in an ELISA reader (Tecan).

Results

Identification of NB-145 through HTS

Using syncytium formation assay and sandwich ELISA-based HTS techniques, a chemical library from Nanosyn Corporation consisting of 46,640 compounds at a single dose (25 μg/ml) has been screened. These compounds are “drug-like” molecules which were rationally pre-selected to form a “universal” library that covers the maximum pharmacophore diversity with the minimum number of compounds. One compound, termed NB-145 at this concentration completely inhibited HIV-1 mediated syncytium formation and the six-helix bundle formation between the gp41 N-peptide N36 and C-peptide C34, suggesting that this compound may inhibit HIV-1 infection by blocking gp41-medaited membrane fusion. Therefore, this compound may be used as a lead compound for identification of more potent HIV-1 fusion inhibitors.

Identification of NB-206 and Its Analogs Which Have Potent Inhibitory Activity on Infection by Laboratory-Adapted HIV-1 Strain IIIB

Based on the chemical structure of NB-145, we searched the chemical database from Chembridge Corporation and found 73 compounds with similar structure of NB-145. We thus purchased these compounds and tested their inhibitory activity on: 1) HIV-1 replication (p24 production); 2) HIV-1-mediated cytopathic effect (CPE); and 3) HIV-1 Env-induced cell-cell fusion; and their cytotoxicity to MT-2 cells. Based the values of CC₅₀ (concentration for 50% cytotoxicity) and IC₅₀ (concentration for 50% inhibition), the selectivity index (SI) was calculated. As shown in Table 2, one of the compounds, designated NB-206, is most potent in inhibiting HIV-1 replication (IC₅₀=19 nM) and HIV-1-mediated cell-cell fusion (IC₅₀=<0.667 μM) with a SI of 981. Besides NB-206, other 20 compounds with identical parent structure of NB-206 also have potent inhibitory activity against HIV-1 infection with IC₅₀ ranging from 87 to 943 nM and SI ranging from 48 to >1778. Most of these active anti-HIV-1 compounds had low cytotoxicity.

TABLE 2
Anti-HIV-1 activity and cytotoxicity of NB-206 and analogs

| | EC50 (μM) (M ± SD) for inhibition of | | | Cytotoxicity | SI |
|---|---|---|---|---|---|
| Compound | Cell fusion | p24 | CPE | CC50 (μM) (M ± SD) | (CC50/EC50) |
| NB139 | — | — | — | 238.77 ± 14.71 | |
| NB140 | — | 12.11 ± 2.73 | 63.41 ± 1.28 | 126.05 ± 19.16 | 10.41 |
| NB145 | 1.77 ± 0.09 | 0.18 ± 0.02 | 0.18 ± 0.006 | 47.48 ± 6.37 | 261.5 |
| NB146 | 1.08147 ± 0.00 | 0.299 ± 0.046 | 1.771 ± 0.368 | 33.843 ± 4.439 | 103.15 |
| NB147 | — | 10.43 ± 0.84 | — | 92.308 ± 5.6165 | 8.85 |
| NB148 | 34.868 ± 0.00 | 2.65 ± 9.38 | 2.65 ± 9.38 | >368 | >266.67 |
| NB150 | 0.56 ± 0.05 | 0.308 ± 0.047 | 0.403 ± 0.119 | 38.726 ± 5.8539 | 125.69 |
| NB151 | 43.694 ± 0.00 | 0.667 ± 0.174 | 3.857 ± 0.580 | 124.845 ± 14.732 | 187.17 |
| NB154 | 0.504 ± 0.01 | 0.241 ± 0.00 | 0.723 ± 0.175 | >350.4 | >1454.54 |
| NB156 | 2.656 | 0.779 ± 0.092 | 3.160 ± 1.351 | >399.4 | >470.59 |
| NB158 | — | — | — | >102.4 | |
| NB160 | — | — | — | >376 | |
| NB179 | 3.658 ± 0.154 | 0.501 ± 0.039 | 0.924 ± 0.077 | >616 | >1230.77 |
| NB180 | — | — | — | >411.2 | |
| NB181 | — | 0.218 ± 0.024 | 0.605 ± 0.194 | >387.2 | >1777.78 |
| NB182 | 1.683 ± 0.142 | 0.830 ± 0.024 | 2.252 ± 1.138 | >379.2 | >457.14 |
| NB183 | 16.759 ± 0.457 | 0.323 ± 0.081 | 0.753 ± 0.081 | 173.613 ± 16.2476 | 537.83 |
| NB184 | — | — | — | >483.2 | >28.93 |
| NB185 | — | 76.931 ± 3.522 | 50.314 ± 4.095 | >436.8 | >5.68 |
| NB186 | — | — | — | >440 | |
| NB187 | — | — | — | 362.97 ± 40.11 | |
| NB188 | — | — | — | 196.77 ± 12.99 | |
| NB189 | 4.325 ± 0.078 | 0.984 ± 0.155 | 2.279 ± 0.389 | >414.4 | >421.05 |
| NB190 | — | — | — | >436.8 | |
| NB191 | — | — | — | >348.8 | |
| NB192 | — | — | — | >363.2 | |
| NB193 | — | — | — | >393.6 | |
| NB194 | — | — | — | 207.16 ± 4.40 | |
| NB195 | — | — | — | 154.37 ± 6.93 | |
| NB196 | — | — | — | >336 | |
| NB197 | — | — | — | >340.8 | |
| NB198 | — | — | — | >321.6 | |
| NB199 | — | — | — | >321.6 | |
| NB200 | — | — | — | >412.8 | |
| NB201 | 33.205 ± 0.566 | 10.667 ± 3.092 | — | 118.236 ± 8.5432 | 11.08 |
| NB202 | — | — | — | >336 | |
| NB203 | — | — | — | >412.8 | |
| NB204 | — | — | — | >366.4 | |
| NB205 | — | — | — | >435.2 | |
| NB206 | 0.459 ± 0.05 | 0.019 ± 0.002 | 0.092 ± 0.002 | 18.808 ± 7.4763 | 981.11 |
| NB207 | — | — | — | >464 | |
| NB208 | 6.668 ± 0.042 | 1.836 ± 0.654 | — | 54.649 ± 15.3186 | 29.77 |
| NB209 | — | — | — | 113.36 ± 14.847 | |
| NB210 | — | — | — | >350.4 | |
| NB211 | 68.56 ± 1.83 | 1.133 ± 0.103 | 2.225 ± 0.515 | 93.174 ± 4.4084 | 82.24 |
| NB212 | — | — | — | >326.4 | |
| NB213 | 14.68 ± 0.599 | 0.514 ± 0.128 | 3.702 ± 1.006 | 92.983 ± 12.7544 | 181.04 |
| NB214 | 2.235 ± 0.082 | 0.943 ± 0.144 | 3.526 ± 0.738 | 44.793 ± 1.2505 | 47.5 |
| NB215 | — | 0.943 ± 0.062 | 1.046 ± 0.021 | >328 | >347.83 |
| NB216 | — | — | — | 55.29 ± 10.04 | |
| NB217 | 2.30 ± 0.131 | 0.767 ± 0.219 | 2.365 ± 0.657 | 165.64 ± 17.71 | 163.2 |
| NB218 | 4.076 ± 0.095 | 0.5453 ± 0.119 | 1.138 ± 0.237 | 56.169 ± 9.81 | 103.04 |
| NB219 | 4.304 ± 0.134 | 0.087 ± 0.040 | 0.401 ± 0.067 | 6.868 ± 0.2899 | 78.97 |
| NB220 | — | 3.047 ± 0.106 | 1.887 ± 0.318 | >339.2 | >110.34 |
| NB221 | 7.11 ± 0.24 | 0.46 ± 0.009 | 1.577 ± 0.241 | 34.514 ± 0.4599 | 75.04 |
| NB222 | — | — | — | >328 | |
| NB223 | 19.07 ± 0.49 | 0.868 ± 0.412 | 2.213 ± 0.304 | 84.50 ± 5.0127 | 97.35 |
| NB224 | 5.581 ± 0.043 | 0.298 ± 0.085 | 0.66 ± 0.064 | 29.479 ± 0.852 | 98.86 |
| NB225 | 33.9.9 ± 0.530 | — | — | >385.6 | |
| NB226 | — | 2.12 ± 0.38 | 3.46 ± 0.20 | >320 | |
| NB227 | 10.506 ± 0.082 | 1.265 ± 0.102 | 1.571 ± 0.367 | 24.5 ± 6.0588 | 19.37 |
| NB228 | — | — | — | >396.8 | |
| NB229 | — | — | — | >376 | |
| NB230 | 3.081 ± 0.047 | 0.332 ± 0.095 | 2.157 ± 0.284 | 142.319 ± 0.00 | 428.93 |
| NB231 | 0.982 ± 0.054 | 0.189 ± 0.042 | 0.525 ± 0.126 | 124.089 ± 0.756 | 656.56 |
| NB232 | — | — | — | >393.6 | |
| NB233 | — | — | — | >372.8 | |
| NB234 | — | — | — | >379.2 | |
| NB235 | — | — | — | >377.6 | |
| NB236 | — | — | — | 112.519 ± 8.639 | |
| NB237 | — | — | — | 67.69 ± 14.99 | |
| NB238 | — | — | — | >388.8 | |
| NB239 | — | — | — | >401.6 | |
| NB238 | 36.84 ± 0.00 | 26.68 ± 2.43 | 42.08 ± 0.53 | >393.6 | >14.57 |
| NB239 | — | — | — | >372.8 | |

“—” means that the compounds at 100 μM had <50% inhibition or no detectable inhibitory activity due to the appearance of cytotoxicity.

NB-206 and Its Analogs Inhibit HIV-1 Entry by Blocking Membrane Fusion

A time-of-addition assay was carried out to determine whether NB-206 and its analogs are HIV-1 entry inhibitors. MT-2 cells were incubated with HIV-1_{IIIB} at 37° C. for 0, 1, 2, 3, 4, 6, and 8 hrs, respectively, before addition of NB-206 and NB-231 at 2.5 μM. AZT (0.1 μM) was used as a control. After culture for another 2 hrs, the cells were washed to remove the free-virus and compounds. The supernatants were collected on day 4 post-infection for measurement of p24 production. NB-206 and its analogs inhibited HIV-1 replication when they were added to the cells with virus together, but showed no inhibitory activity if they were added one hour or longer after virus was added to cells. However, AZT was still effective in inhibiting HIV-1 replication even it was added 8 hrs post-infection (FIG. 1).

Fusion between virus and target cell membranes or between HIV-infected cells and uninfected cells is the critical steps of HIV entry into a new target cell. Therefore, it is essential to determine whether NB-206 and its analogs inhibit cell-cell fusion. As shown in FIG. 2, NB-206 and its analogs (NB-231, NB-154, and NB-179) inhibited fusion of HIV-1_{IIIB} infected H9 cells with uninfected MT-2 cells, in dose dependent manner.

NB-206 and Its Analogs Have Potent Inhibitory Activity on Infection by Laboratory-Adapted and Primary HIV-1 Strains

The inhibitory activity of NB-206 and its analogs on infection of MT-2 cells by laboratory-adapted HIV-1 strains and of CEMx174 5.25 M7 cells by primary HIV-1 strains was determined as previously described (23, 38). In addition to HIV-1 IIIB, NB-206 and its analogs (NB-231, NB-154, and NB-179) also inhibited, in dose-dependent manner, infection by other laboratory-adapted HIV-1 strains, including RF, SF2, MN, and AZT-R, a strain resistant to AZT, with IC50 values in nanomolar range (Table 3).

TABLE 3
Inhibitory activity of NB206 and its analogs on
infection by laboratory-adapted HIV-1 strains

| HIV- | | | | |
|---|---|---|---|---|
| 1 | IC₅₀ (μM) (Mean ± SD) | | | |
| strain | NB-206 | NB-231 | NB-154 | NB-179 |
| IIIB | 0.019 ± 0.002 | 0.189 ± 0.042 | 0.241 ± 0.001 | 0.501 ± 0.039 |
| RF | 0.034 ± 0.014 | 0.152 ± 0.029 | 0.152 ± 0.025 | 4.199 ± 0.256 |
| SF2 | 0.261 ± 0.062 | 4.682 ± 0.559 | 0.568 ± 0.084 | 11.911 ± 1.703 |
| MN | 0.174 ± 0.027 | 1.685 ± 0.044 | 0.271 ± 0.013 | 2.925 ± 0.362 |
| AZT- | 0.046 ± 0.004 | 0.823 ± 0.053 | 0.084 ± 0.008 | 4.386 ± 0.471 |
| R | | | | |

The inhibitory activity of NB-206 and its analogs on infection by primary HIV-1 isolates with distinct subtypes (clades A, B, C, E, F, G, and group o) and biotype (R5, X4, and R5/X4) was determined as previously described (24). As shown in Table 4, NB-206 and its analogs had potent inhibitory activity on infection by primary HIV-1 isolates with IC₅₀ values in nanomolar range. These data suggest that NB-206 and its analogs have potent antiviral activity against a broad spectrum of HIV-1 strains.

TABLE 4
Inhibitory activity of NB206 and its analogs on
infection by primary HIV-1 strains

| HIV-1 isolate | | | | |
|---|---|---|---|---|
| (subtype, | EC50 (μM) (Mean ± SD) | | | |
| coreceptor usage) | NB206 | NB231 | NB154 | NB179 |
| 94UG103 (clade A, X4R5) | 0.004 ± 0.001 | 0.125 ± 0.013 | 0.067 ± 0.005 | 0.088 ± 0.047 |
| 92US657 (clade B, R5) | 0.216 ± 0.054 | 1.063 ± 0.344 | 1.624 ± 0.535 | 0.847 ± 0.356 |
| 93MW959 (clade C, R5) | 0.097 ± 0.002 | 0.876 ± 0.002 | 1.944 ± 0.180 | 1.869 ± 0.155 |
| 92UG001 (clade D, X4) | 0.188 ± 0.053 | 0.190 ± 0.002 | 0.309 ± 0.025 | 0.613 ± 0.099 |
| 92THA009 (clade E, R5) | 0.075 ± 0.022 | 0.217 ± 0.058 | 0.632 ± 0.027 | 6.207 ± 0.987 |
| 93BR020 (clade F, X4R5) | 0.084 ± 0.036 | 0.139 ± 0.033 | 0.076 ± 0.028 | 0.143 ± 0.056 |
| RU570 (clade G, R5) | 0.017 ± 0.004 | 0.143 ± 0.061 | 0.296 ± 0.038 | 0.431 ± 0.122 |
| BCF02 (clade O, R5) | 0.073 ± 0.012 | 0.226 ± 0.003 | 0.571 ± 0.069 | 0.886 ± 0.242 |

NB-206 and Its Analogs Interfere with the gp41 Six-Helix Bundle Formation

Subsequently, the effect of NB-206 and its analogs on the gp41 six-helix bundle formation, a critical conformational change during HIV-1 fusion with the target cells, was determined. A model system of the gp41 six-helix bundle was established by mixing the N— and C-peptides at equal molar concentrations (16). This model gp41 core structure can be detected by sandwich ELISA using a conformation-specific mAb, NC-1 (27,29). Using this system, the inhibitory activity of NB-206 and its analogs on the gp41 six-helix bundle formation was tested. As shown in FIG. 3, NB-206 and its analogs (NB-231, NB-154, and NB-179) significantly inhibited the six-helix bundle formation between N36 and C34 in a dose-dependent manner. The IC50 (μM) values of NB-206, NB-231, NB-154, and NB-179 are: 0.83±0.03, 0.93±0.33, 1.56±0.12, and 2.51±0.27, respectively. These results suggest that NB-206 and its analogs may bind to a component in the gp41 coiled coil domains and interfere with the association between the gp41 NHR and CHR regions.

Discussion of the Results

During the past 20 years, one of the greatest progresses in HIV/AIDS research is the development of anti-HIV drugs (39). So far, 20 anti-HIV drugs have been approved by the US FDA and more drug candidates are in the pipelines (40). Most of these drugs are targeted to the HIV-1 reverse transcriptase and protease. Only one of them, Fuzeon (T-20), targets the viral envelope glycoprotein gp41 (14,38,41,42). T-20 (41), like other peptides derived from the HIV-1 gp41 CHR region, such as SJ-2176 (11,43) and C34 (17), inhibits HIV-1 fusion and entry. It has shown great promise against HIV replication in clinical trials (14,44). However, it has two major limitations: lack of oral availability (delivered by subcutaneous injection) and high cost of production (40). Thus, development of small molecule HIV-1 fusion inhibitors is urgently needed.

It was previously reported that the identification of several small molecule HIV-1 fusion inhibitors, ADS-J1, NB-2 and NB-64, through screening using cell-based HIV-1 fusion assays, a sandwich ELISA using a conformation-specific mAb NC-1 and computer-aided molecular docking techniques (21,24,27). However, these compounds may not be good lead compounds since their anti-HIV-1 activities are in micromolar levels. Therefore, it was necessary to screen more chemical libraries to identify more potent small molecule HIV-1 entry inhibitors targeting gp41.

Using a two-step screening assays (Syncytium formation assay and ELISA for 6-HB formation), one HIV-1 fusion inhibitor, NB-145, was identified from a chemical library consisting of 46,640 “drug-like” compounds.

Then we purchased 73 compounds structurally analogous to NB-145 from a chemical company (Table 1) and tested their inhibitory activity against HIV-1 replication, HIV-1-mediated CPE and cell-cell fusion, as well as the gp41 6-HB formation. We identified a compound NB-206 with highly potent anti-HIV-1 activity (IC50 at low nanomolar level) and relatively low cytotoxicity and high SI (approximately 1000). We also identified another set of 20 compounds with similar structure of NB-206 with potent inhibitory activity against HIV-1 IIIB infection with IC50 values in nM range and high SI (some reach to >1500) (Table 2) In addition to HIV-1 IIIB, NB-206 and its analogs are also highly potent in inhibiting infection by other laboratory-adapted HIV-1 strain, including RF, SF2, MN and AZT-R, a strain resistant to AZT (Table 3). They are effective against infection by representative primary isolates with distinct subtypes and biotypes (Table 4). NB-206 and its analogs have potent inhibitory activity against 6-HB formation, suggesting that these small molecule HIV-1 entry fusion inhibitors block HIV-1 fusion by targeting gp41.

NB-206 and its analogs have “drug-like” properties based on the Lipinski's “rule of five” (45), i.e., molecular weight<500 daltons, the calculated CLogP<5, H-bond donors<5 and H-bond acceptors<10. Therefore, these compounds may have good permeability and bioavailability.

Although design of small molecule organic compounds to block protein-protein interaction is a challenging approach for drug development (46), identification of such inhibitors have been reported (47-49). Recently, a small molecule HIV-1 entry inhibitor, BMS-378806, was discovered (50). This compound with a molecular weight of 406.5 is very potent to block interaction between the viral envelope glycoprotein gp120 and the cellular receptor CD4. This suggests that a small molecule compound, if bound to a “hot spot” in a protein, such as a hydrophobic pocket, may effectively block protein-protein interaction. The deep hydrophobic pocket on the surface of the gp41 internal trimer formed by the NHR domains has been recognized as a “hot spot” since it may play important roles in the formation and the stability of the gp41 six-helix bundle (20,51). NB-206 and its analogs may bind to the gp41 pocket to block the formation of the fusion-active gp41 core.

NB-206 and its analogs have broad anti-HIV-1 activity against distinct HIV-1 strains and a specificity to target gp41. Thus, NB-206 and its analogs may be used as leads for designing novel anti-virus compositions, particularly, more potent small molecule HIV-1 entry inhibitors as a new class of anti-HIV-1 drugs.

### REFERENCES

1. Hunter, E. 1997. Viral entry and receptor. In Retroviruses. J. M. Coffin, S. H. Hughes, and H. E. Varmus, editors. Cold Spring Harbor Laboratory Press, Cold Spring Harbor, N.Y. 71-119.
2. Sattentau, Q. J. and J. P. Moore. 1991. Conformational changes induced in the human immunodeficiency virus envelope glycoprotein by soluble CD4 binding. J. Exp. Med. 174: 407-415.
3. Sattentau, Q. J., J. P. Moore, F. Vignaux, F. Traincard, and P. Poignard. 1993. Conformational changes induced in the envelope glycoproteins of the human and simian immunodeficiency viruses by soluble receptor binding. J. Virol. 67: 7383-7393.
4. Moore, J. P. and R. W. Sweet. 1993. The HIV gp120-CD4 interaction: A target for pharmacological or immunological intervention? Perspectives in Drug Discovery and Design 1:235-250.
5. Berger, E. A. 1998. HIV entry and tropism. When one receptor is not enough. Adv. Exp. Med. Biol. 452:151-157.
6. Moore, J. P. 1997. Coreceptors: implications for HIV pathogenesis and therapy. Science 276:51-52.
7. Chan, D. C., D. Fass, J. M. Berger, and P. S. Kim. 1997. Core structure of gp41 from the HIV envelope glycoprotein. Cell 89:263-273.
8. Melikyan, G. B., R. M. Markosyan, H. Hemmati, M. K. Delmedico, D. M. Lambert, and F. S. Cohen. 2000. Evidence that the transition of HIV-1 gp41 into a six-helix bundle, not the bundle configuration, induces membrane fusion. J. Cell Biol. 151:413-424.
9. Wild, C., T. Greenwell, and T. Matthews. 1993. A synthetic peptide from HIV-1 gp41 is a potent inhibitor of virus-mediated cell-cell fusion. AIDS Res. Hum. Retrov. 9:1051-1053.
10. Wild, C., T. Oas, C. McDanal, D. Bolognesi, and Matthews. T. 1992. A synthetic peptide inhibitor of human immunodeficiency virus replication: correlation between solution structure and viral inhibition. Proc. Natl. Acad. Sci. USA. 89: 10537-10541.
11. Jiang, S., K. Lin, N. Strick, and A. R. Neurath. 1993. HIV-1 inhibition by a peptide. Nature 365:113.
12. Hardy, H. and P. R. Skolnik. 2004. Enfuvirtide, a new fusion inhibitor for therapy of human immunodeficiency virus infection. Pharmacotherapy 24:198-211.
13. Lazzarin, A. 2005. Enfuvirtide: the first HIV fusion inhibitor. Expert. Opin. Pharmacother. 6:453-464.
14. Lalezari, J. P., K. Henry, M. O'Hearn, J. S. Montaner, P. J. Piliero, B. Trottier, S. Walmsley, C. Cohen, D. R. Kuritzkes, J. J. Eron, Jr., J. Chung, R. DeMasi, L. Donatacci, C. Drobnes, J. Delehanty, and M. Salgo. 2003. Enfuvirtide, an HIV-1 fusion inhibitor, for drug-resistant HIV infection in north and south America. N. Engl. J. Med. 348:2175-2185.
15. Kilby, J. M. and J. J. Eron. 2003. Novel therapies based on mechanisms of HIV-1 cell entry. N. Engl. J Med. 348:2228-2238.
16. Lu, M., S. C. Blacklow, and P. S. Kim. 1995. A trimeric structural domain of the HIV-1 transmembrane glycoprotein. Nat. Struct. Biol. 2: 1075-1082.
17. Lu, M. and P. S. Kim. 1997. A trimeric structural subdomain of the HIV-1 transmembrane glycoprotein. J. Biomol. Struct. Dyn. 15:465-471.
18. Weissenhorn, W., A. Dessen, S. C. Harrison, J. J. Skehel, and D. C. Wiley. 1997. Atomic structure of the ectodomain from HIV-1 gp41. Nature 387:426-430.
19. Markosyan, R. M., F. S. Cohen, and G. B. Melikyan. 2003. HIV-1 envelope proteins complete their folding into six-helix bundles immediately after fusion pore formation. Mol. Biol. Cell 14:926-938.
20. Chan, D. C., C. T. Chutkowski, and P. S. Kim. 1998. Evidence that a prominent cavity in the coiled coil of HIV type 1 gp41 is an attractive drug target. Proc. Natl. Acad. Sci. U.S.A. 95:15613-15617.
21. Debnath, A. K., L. Radigan, and S. Jiang. 1999. Structure-based identification of small molecule antiviral compounds targeted to the gp41 core structure of the human immunodecifiency virus type 1. J. Med. Chem. 42:3203-3209.
22. Jiang, S. and A. K. Debnath. 2000. A salt bridge between an N-terminal coiled coil of gp41 and an antiviral agent targeted to the gp41 core is important for anti-HIV-1 activity. Biochem. Biophys. Res. Commun. 270:153-157.
23. Zhao, Q., J. T. Ernst, A. D. Hamilton, A. K. Debnath, and S. Jiang. 2002. XTT formazan widely used to detect cell viability inhibits HIV type 1 infection in vitro by targeting gp41. AIDS Res. Hum. Retrov. 18:989-997.
24. Jiang, S., H. Lu, S. Liu, Q. Zhao, Y. He, and A. K. Debnath. 2004. N-substituted pyrrole derivatives as novel human immunodeficiency virus type 1 entry inhibitors that interfere with the gp41 six-helix bundle formation and block virus fusion. Antimicrob. Agents Chemother. 48:4349-4359.
25. Jiang, S., L. Radigan, and L. Zhang. 2000. A convenient cell fusion assay for rapid screening for HIV entry inhibitors. Proc. SPIE 3926:212-219.
26. Lu, H., Q. Zhao, Z. Xu, and S. Jiang. 2003. Automatic quantitation of HIV-1 mediated cell-to-cell fusion with a digital image analysis system (DIAS): application for rapid screening of HIV-1 fusion inhibitors. J. Virol. Methods 107:155-161.
27. Jiang, S., K. Lin, L. Zhang, and A. K. Debnath. 1999. A screening assay for antiviral compounds targeted to the HIV-1 gp41 core structure using a conformation-specific monoclonal antibody. J. Virol. Methods 80:85-96.
28. Liu, S., L. Boyer-Chatenet, H. Lu, and S. Jiang. 2003. Rapid and automated fluorescence-linked immunosorbent assay for high throughput screening of HIV-1 fusion inhibitors targeting gp41. J. Biomol. Screening 8:685-693.
29. Jiang, S., K. Lin, and M. Lu. 1998. A conformation-specific monoclonal antibody reacting with fusion-active gp41 from the HIV-1 envelope glycoprotein. J. Virol. 72:10213-10217.
30. Hsu, M., J. M. Harouse, A. Gettie, C. Buckner, J. Blanchard, and C. Cheng-Mayer. 2003. Increased mucosal transmission but not enhanced pathogenicity of the CCR5-tropic, simian AIDS-inducing simian/human immunodeficiency virus SHIV(SF162P3) maps to envelope gp120. J. Virol. 77:989-998.
31. Eckert, D. M., V. N. Malashkevich, L. H. Hong, P. A. Carr, and P. S. Kim. 1999. Inhibiting HIV-1 entry: discovery of D-peptide inhibitors that target the gp41 coiled-coil pocket. Cell 99:103-115.
32. Nakashima, H., M. Masuda, T. Murakami, Y. Koyanagi, A. Matsumoto, N. Fujii, and N. Yamamoto. 1992. Anti-human immunodeficiency virus activity of a novel synthetic peptide, T22 ([Tyr-5,12, Lys-7]polyphemusin II): a possible inhibitor of virus-cell fusion. Antimicrob. Agents. Chemother. 36:1249-1255.
33. Murakami, T., T. Nakajima, Y. Koyanagi, K. Tachibana, N. Fujii, H. Tamamura, N. Yoshida, M. Waki, A. Matsumoto, O. Yoshie, T. Kishimoto, N. Yamamoto, and T. Nagasawa. 1997. A small molecule CXCR4 inhibitor that blocks T cell line-tropic HIV-1 infection. J. Exp. Med. 186:1389-1393.
34. Jiang, S., K. Lin, and A. R. Neurath. 1991. Enhancement of human immunodeficiency virus type-1 (HIV-1) infection by antisera to peptides from the envelope glycoproteins gp120/gp41. J. Exp. Med. 174: 1557-1563.
35. Chou, T.-C. and M. P. Hayball CalcuSyn: Windows Software for Dose Effect Analysis 1991. Ferguson, Mo. 63135, USA, BIOSOFT.
36. Prince, A. M., B. Horowitz, L. Baker, R. W. Shulman, H. Ralph, J. Valinsky, A. Cundell, B. Brotman, W. Boehle, F. Rey, M. Piet, H. Reesink, N. Lelie, M. Tersmette, F. Miedema, L. Barbosa, G. Nemo, C. L. Nastala, J. S. Allan, D. R. Lee, and J. W. Eichberg. 1988. Failure of a human immunodeficiency virus (HIV) immune globulin to protect chimpanzees against experimental challenge with HIV. Proc. Natl. Acad. Sci. U.S.A. 85:6944-6948.
37. Neurath, A. R., S. Jiang, N. Strick, K. Lin, Y.-Y. Li, and A. K. Debnath. 1996. Bovine β-lactoglobulin modified by 3-hydroxyphthalic anhydride blocks the CD4 cell receptors for HIV-1. Nature Med. 2:230-234.
38. Huang, L., L. Zhang, and C. H. Chen. 2003. Potential drug targets on the HIV-1 envelope glycoproteins, gp120 and gp41. Curr. Pharm. Des. 9:1453-1462.
39. Fauci, A. S. 2003. HIV and AIDS: 20 years of science. Nat. Med. 9:839-843.
40. Pomerantz, R. J. and D. L. Horn. 2003. Twenty years of therapy for HIV-1 infection. Nat. Med. 9:867-873.
41. Wild, C. T., D. C. Shugars, T. K. Greenwell, C. B. McDanal, and T. J. Matthews. 1994. Peptides corresponding to a predictive alpha-helical domain of human immunodeficiency virus type 1 gp41 are potent inhibitors of virus infection. Proc. Natl. Acad. Sci. USA. 91: 9770-9774.
42. Chen, C. H., T. J. Matthews, C. B. McDanal, D. P. Bolognesi, and M. L. Greenberg. 1995. A molecular clasp in the human immunodeficiency virus (HIV) type 1 TM protein determines the anti-HIV activity of gp41 derivatives: implication for viral fusion. J. Virol. 69: 3771-3777.
43. Jiang, S., K. Lin, N. Strick, and A. R. Neurath. 1993. Inhibition of HIV-1 infection by a fusion domain binding peptide from the HIV-1 envelope glycoprotein GP41. Biochem. Biophys. Res. Commun. 195:533-538.
44. Kilby, J. M., J. P. Lalezari, J. J. Eron, M. Carlson, C. Cohen, R. C. Arduino, J. C. Goodgame, J. E. Gallant, P. Volberding, R. L. Murphy, F. Valentine, M. S. Saag, E. L. Nelson, P. R. Sista, and A. Dusek. 2002. The safety, plasma pharmacokinetics, and antiviral activity of subcutaneous enfuvirtide (T-20), a peptide inhibitor of gp41-mediated virus fusion, in HIV-infected adults. AIDS Res. Hum. Retrov. 18:685-693.
45. Lipinski, C. A., F. Lombardo, B. W. Dominy, and P. J. Feeney. 2001. Experimental and computational approaches to estimate solubility and permeability in drug discovery and development settings. Adv. Drug Deliv. Rev. 46:3-26.
46. Cochran, A. G. 2001. Protein-protein interfaces: mimics and inhibitors. Curr. Opin. Chem. Biol. 5:654-659.
47. Toogood, P. L. 2002. Inhibition of protein-protein association by small molecules: approaches and progress. J. Med. Chem. 45:1543-1558.
48. Ockey, D. A. and T. R. Gadek. 2002. Inhibitors of protein-protein interactions. Expert Opinion on Therapeutic Patents 12:393-400.
49. Gadek, T. R. and J. B. Nicholas. 2003. Small molecule antagonists of proteins. Biochem. Pharmacol. 65:1-8.
50. Lin, P. F., W. Blair, T. Wang, T. Spicer, Q. Guo, N. Zhou, Y. F. Gong, H. G. Wang, R. Rose, G. Yamanaka, B. Robinson, C. B. Li, R. Fridell, C. Deminie, G. Demers, Z. Yang, L. Zadjura, N. Meanwell, and R. Colonno. 2003. A small molecule HIV-1 inhibitor that targets the HIV-1 envelope and inhibits CD4 receptor binding. Proc Natl Acad Sci USA 100:11013-11018.
51. Dwyer, J. J., A. Hasan, K. L. Wilson, J. M. White, T. J. Matthews, and M. K. Delmedico. 2003. The hydrophobic pocket contributes to the structural stability of the N-terminal coiled coil of HIV gp41 but is not required for six-helix bundle formation. Biochemistry 42:4945-4953.

1. An anti-viral composition comprising an effective amount of a compound comprising formula (I) or a pharmaceutically acceptable salt thereof: 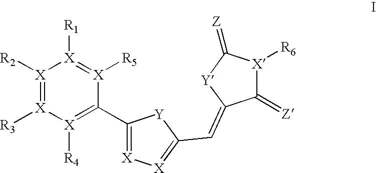
a) wherein at least one of R₁, R₂, R₃, R₄, R₅, or R₆ contains COOH or other acidic groups;
b) wherein X, X′, Y and Y′ is C, N, O or S;
c) wherein Z and Z, is O or S; and,
d) wherein R₁—R₆ are independently selected from the group consisting of
H,
alkyl,
cycloalkyl,
alkenyl,
alkynyl,
aryl,
arylalkyl,
alkylaryl,
heterocyclyl,
tetrazolyl,
adamantyl
halogen,
trifluoromethyl,
OH,
CN,
NO₂,
OR₇ where R₇ is alkyl, aryl or arylalkyl,
COOR₈ where R₈ is H or alkyl,
SO₃R₉ where R₉ is H or alkyl,
SO₂NHR₁₀ where R₁₀ is H or alkyl, and
CONHR₁₁ where R₁₁ is H or alkyl.

2. The composition of claim 1 wherein X and X′, Y and Y′ are either O or S, the bond with the next atom if C, will be a single bond and O or S will be unsubstituted and when X and X′,Y and Y′ are N, it is either unsubstituted or substituted with H, alkyl, cycloalkyl, alkenyl, alkynyl, aryl, arylalkyl or heterocyclyl groups.

3. The composition of claim 1 wherein X is a carbon, X′ is nitrogen, Y and Z′ are oxygen, Y′ and Z are sulfur.

4. The composition of claim 3 wherein R₁—R₆ is independently selected from the group consisting of
H,
alkyl,
cycloalkyl,
alkenyl,
alkynyl,
aryl,
arylalkyl,
alkylaryl,
heterocyclyl,
tetrazolyl,
adamantyl,
halogen,
trifluoromethyl,
OH,
CN,
NO₂,
OR₇ where R₇ is alkyl, aryl or arylalkyl,
COOR₈ where R₈ is H or alkyl,
SO₃R₉ where R₉ is H or alkyl,
SO₂NHR₁₀ where R₁₀ is H or alkyl, and
CONHR₁₁ where R₁₁ is H or alkyl.

5. The composition of claim 4 wherein the group alkyl is substituted with straight or branched alkyl chains carrying 1 to 6 carbon atoms.

6. The composition of claim 4 wherein alkyl is methyl, ethyl, n-propyl, i-propyl, n-butyl, i-butyl, tert-butyl, pentyl or hexyl.

7. The composition of claim 4 wherein alkenyl is substituted with straight or branched alkenyl chains carrying 2 to 6 carbon atoms.

8. The composition of claim 4 wherein the alkenyl is vinyl, 1-propenyl, 2-propenyl, i-propenyl, butenyl, or its isomers, pentenyl or hexenyl.

9. The composition of claim 4 wherein alkynyl is substituted with straight or branched alkynyl chains carrying 2 to 6 carbon atoms.

10. The composition of claim 4 wherein the alkynyl group is ethynyl, propynyl or its isomers, butynyl or its isomers, pentynyl or hexynyl.

11. The composition of claim 4 wherein the halogen group is chloro, bromo, fluoro, or iodo.

12. The composition of claim 4 wherein substituents of alkyl, alkenyl and alkynyl are selected from one or more of the following: amino, cyano, halogen, hydroxy, alkoxy, aryloxy, aryl, heterocyclyl, carboxy, nitro, alkyl sulfonyl, aryl sulfonyl, thio, alkyl thio, or aryl thio.

13. The composition of claim 4 wherein cycloalkyl is substituted with cycloalkyl groups containing 3 to 6 carbon atoms.

14. The composition of claim 4 wherein the cycloalkyl is cyclopropyl, cyclobutyl, cyclopentyl, cyclohexyl.

15. The composition of claim 4 wherein the cycloalkyl is benz-fused to an aromatic cyclic group.

16. The composition of claim 4 wherein the aryl is substituted with phenyl or napthyl.

17. The composition of claim 16 wherein the phenyl or napthyl group is optionally substituted with amino, cyano, halogen, hydroxyl, alkoxy, carboxy, nitro, thio, alkyl, or trifluoromethyl.

18. The composition of claim 4 wherein the group heterocyclic is optionally substituted with saturated, partially saturated, or aromatic cyclics, which contain one or more heteroatoms selected from nitrogen, oxygen or sulfur.

19. The composition of claim 4 wherein the compound is benz-fused to a substituted aromatic cyclic or heterocycles.

20. The composition of claim 18 wherein the heterocyclic group is quinolinyl, pyridyl, indolyl, furyl, oxazolyl, thienyl, triazolyl, pyrazolyl, imidazolyl, benzothiazolyl, benzimidazolyl, piperzinyl, or benzothiazolyl.

21-33. (canceled)
